(19) United States
(12) Patent Application Publication
(10) Pub. No.: US 2003/0092972 A1
(43) Pub. Date: May 15, 2003

(54) Telephone- and network-based medical triage system and process

(76) Inventors: David Alejandro Mantilla, Bronx, NY (US); Javier Jerry Lugo, White Plains, NY (US)

Correspondence Address: DAVID ALEJANDRO MANTILLA, 3967 Sedgwick Avenue Apt #13C, Bronx, NY, 10463, US

(21) Appl. No.: 10/040,113

(22) Filed: Nov. 9, 2001

(51) Int. Cl. A61B 5/00

(52) U.S. Cl. 600/300; 128/900

(57) ABSTRACT

The present solution to the medical triage problem is computer automated telephone triage system that gives customized medical advice to patients of doctors who have registered with the service. There are two main modes of operation: a nurse-assisted mode, and a fully-automated mode. In the nurse-assisted system, a person desiring medical information calls the triage system and is connected to a nurse. The nurse asks a few questions and determines the caller's doctor, main symptom, and whether a high-risk medical condition exists. If there exists a serious condition then the nurse give appropriate instructions according to a triage protocol, Otherwise, the nurse connects the caller to the VoiceTriage program. This program plays back an audio message recorded by the caller's doctor giving the caller specific medical instructions to treat the caller's symptom. The fully-automated mode replaces the nurse-caller interaction with a computer-caller one. A main object of the present invention is to provide a patient with medical advice of his own doctor, in doctor's own voice to minimize confusion, increase quality of service and increase patient satisfaction with a triage systems and medical answering services.

### CROSS-REFERENCE TO RELATED APPLICATIONS

Not Applicable.

### BACKGROUND—STATEMENT REGARDING FEDERALLY SPONSORED RESEARCH OR DEVELOPMENT

Not Applicable.

### BACKGROUND

1. Field of Invention

The present invention relates to systems for giving medical advice to the general public over networks.

2. Description of Prior Art

Of the telephone calls that a doctor's office receives in a given 24-hour period, approximately 3% are emergencies, 45% are about sick children who need an appointment, and the remaining 52% can be treated at home with appropriate medical instructions. Doctors do not want to bothered with the majority of non-emergency calls during after-hours when they are on-call but no longer holding office hours. However, doctors must respond to medical emergencies regardless of the time of day. Doctors need a triage system that will free them from dealing with non-emergencies during after-hours while being accessible to patients in case of medical emergencies. The triage problem is more acute with pediatricians since their patients are minors whose worried parents hound the pediatrician with questions.

One prior attempt at a solution to the medical triage problem can be found in U.S. Pat. No. 5,764,923 to Tallman, et al., Jun. 9, 1998. Their solution relates generally to a system and process for managing health care for payers, patients, and providers. More particularly, it is a system and process which interfaces with health plan beneficiaries who have decided to seek health care services from a doctor and/or some other type of health care provider. These calls are answered by nurses and/or other types of health care professionals, who use proprietary information tools and processes to help patients assess their health needs and then select appropriate care. However, this “telephone triage” system does not adequately address the problem we are focusing on and that affects many doctors: how to handle the large number of incoming phone calls to a doctor's office on a daily basis. Instead, the aforementioned solution borders on a doctor referral service.

Another prior attempt at a solution to the triage problem is called Ask-A-Nurse, wherein a group of nurses provide health information by telephone around-the-clock. A person with a medical problem calls an 800 number and describes the problem to the nurse. The nurse uses a computer for general or diagnostic information on the ailment or complaint mentioned by the caller. The nurse may then refer the caller to a doctor from a computerized referral list for a contracting hospital or group of hospitals. Client hospitals contract with Ask-A-Nurse to provide patient referrals. A managed care option called Personal Health Advisor is similar and adds the capability for the caller to hear prerecorded messages on health topics 24 hours a day. Several problems exist with these prior medical advice systems. First, these systems have high costs associated with having a nurse answer each telephone call. Second, the caller may have to belong to a participating health plan to utilize the service. Third, if for some reason all nurses on a particular shift happen to be busy and the caller has an emergency condition (that is not known by the caller to be an emergency), precious time in getting emergency services may be lost during the delay.

A fourth and important problem is that often patients do not trust the medical advice given to them by strangers over the phone. That is, patients have no personal connection to nurses they have never met and who are often located miles away from the patient's neighborhood. This is especially true in the field of pediatrics in which worried parents many times refuse to talk over the phone to any one except their child's pediatrician. Parents will repeatedly ignore the advice given to them by the nurses working at a particular doctor's office only to hear the same advice recited to them by their child's doctor moments later.

Many doctors have answering services to screen their phone calls during after-hours; that is, during the hours which their office is closed. Doctors pay hundreds of dollars a month for untrained medical professionals to take phone messages during after-hours. No medical advice is given during these sessions. If there is a medical emergency, the doctor is paged accordingly. Otherwise the doctor receives a list of messages next time he arrives at the office. There are more advanced, and more expensive, answering services in which medically trained staff answer phones and follow a predefined telephone triage protocol. These protocols help determine whether to classify an incoming call as an emergency call, one that requires a next-day appointment, or a call that can be treated at home if the caller is given appropriate medical instructions. Although these answering services are a step in the right direction, the staff often does not reliably document a telephone conversation, or the doctor disagrees with the (generic) medical advice given to her patient. Improved consistency in documentation of incoming calls and advice given, as well as some customization of the medical advice dispensed to patients on the part of the doctor, is left to be desired.

Another prior health system was developed by InterPractice Systems which provides a computerized service that answers health care questions and advises people in their homes. A health maintenance organization (HMO) may provide this service to its members in a particular geographic area. To get advice at home, an HMO member connects a toaster-sized box to a telephone and calls a toll-free 800 number. Using a keyboard that is part of the box, the user answers questions displayed on a screen of the box relating to the user's symptoms. Depending on the answers, the user might be told to try a home remedy, be called by a nurse or doctor, or be given an appointment to be examined. A limitation of this system is the additional expense of the electronics box, which could either be purchased by the user for approximately $300 or purchased by the health organization with the expense to be passed on to the users. Another limitation is that this service is directed to members of a particular contracting health organization, such as an HMO. What is desired is a system that does not require additional hardware for the basic service, but that utilizes the existing communication network. The desired system should be available for use by any person, not just members of a certain organization.

U.S. Pat. No. 4,838,275 discloses a device for a patient to lay on or sit in having electronics to measure multiple parameters related to a patient's health. These parameters are electronically transmitted to a central surveillance and control office where a highly trained observer interacts with the patient. The observer conducts routine diagnostic sessions except when an emergency is noted or from a patient-initiated communication. The observer determines if a non-routine therapeutic response is required, and if so facilitates such a response. Highly trained people are needed by this system along with the special measurement apparatus (embedded in a bed or chair).

Other prior attempts at a health care solution are typified by U.S. Pat. No. 5,012,411 which describes a portable self-contained apparatus for measuring, storing and transmitting detected physiological information to a remote location over a communication system. The information is evaluated by a physician or other health professional. As before, highly trained people are necessary to utilize such an apparatus.

Several services to provide medical or pharmaceutical advice are now available via “1-900” telephone numbers, e.g., “Doctors by Phone.” These services are available 24 hours a day and 7 days a week. A group of doctors, including some specialties, is available to answer questions about health care or medical conditions for people anywhere in the United States who call the “1-900” telephone of one of the services. A group of registered pharmacists answers questions about medications for the “1-900” pharmaceutical service. Again, many patients would rather receive their own doctor's advice than an unfamiliar doctor's advice.

### SUMMARY OF THE INVENTION

The present solution to the medical triage problem is a computer automated telephone triage system that gives customized medical advice to patients of doctors who have registered with the service. To provide service for all callers, the advice of a “head doctor” can be given to patients not registered with the system. There are two main modes of operation: a nurse-assisted mode, and a fully-automated mode. In the nurse-assisted system, a person desiring medical information calls the triage system and is connected to a nurse. The nurse asks a few questions and determines the caller's doctor, main symptom, and whether a high-risk medical condition exists. If there exists a serious condition then the nurse gives appropriate instructions according to a triage protocol. Otherwise, the nurse connects the caller to the VoiceTriage program. This program plays back an audio message recorded by the caller's doctor giving the caller specific medical instructions to treat the caller's symptom.

The fully-automated mode replaces the nurse-caller interaction with a computer-caller one. A person seeking medical advice dials a given phone number and is connected to the FA-VoiceTriage program. Both the FA-VoiceTriage and the nurse-assisted VoiceTriage programs recognize human speech.

A main object of the present invention is to provide a patient with medical advice of his own doctor, in doctor's own voice to minimize confusion, increase quality of service and increase patient satisfaction with a triage systems and medical answering services. The FA-VoiceTriage system is customizable and allows for the personalization of the computer-driven dialogue. For example, like the nurse-assisted VoiceTriage system, doctors can add their own high-risk questions to the standard computer-driven dialogue if they wish. Furthermore, the system can respond to and understand other languages besides English, such as French and German.

The attainment of the foregoing and related objects, advantages and features of the invention should be more readily apparent to those skilled in the art, after review of the following more detailed description of the invention, taken together with the drawings, in which:

### BRIEF DESCRIPTION OF DRAWINGS

FIG. 1 depicts the principle elements of complete system in which the preferred embodiment of computerized telephone triage system (CTTS) of the present invention operates and the relationship of the elements of the system to each other.

FIGS. 2a, 2b, and 2c are a top-level flow diagram of a nurse-assisted embodiment of the CTTS system of FIG. 1.

FIGS. 3a and 3b are a top-level flow diagram of a fully automated embodiment of the CTTS system of FIG. 1.

FIG. 4 is a flow diagram of the doctor menu 110 defined in FIG. 3b.

FIG. 5 is a flow diagram of the symptom menu 112 defined in FIG. 3b.

FIG. 6 is a flow diagram of the ask-question function 114 defined in FIG. 3b.

FIG. 7 is a flow diagram of the doctor-symptom menu 54 defined in FIG. 2c.

FIG. 8 is a flow diagram of the playback function 99 defined in FIG. 2a.

FIG. 9 is a flow diagram of the generate file function 226 defined in FIG. 2a.

FIGS. 10a and 10b are a flow diagram of the generate VXML function 310 defined in FIG. 9.

FIGS. 11a and 11b are a flow diagram of the generate HTML function 308 defined in FIG. 9.

FIG. 12 is a flow diagram of a computer-generated VXML file.

FIG. 13 is a flow diagram of the error-checking process 4096 defined in FIG. 12.

FIG. 14 is a block diagram of various tables of a database 626.

FIGS. 15a and 15b are representations of HTML files for login and questionnaire windows used by nurse 606 during NATT program.

FIGS. 16a and 16b are representations of HTML files displaying the doctor's audio message and video recording during the FATT program.

FIG. 17 is a block diagram showing elements of a complete system of an alternative embodiment of the present invention and the relationship of the elements of the system to each other.

### DETAILED DESCRIPTION OF INVENTION

The following detailed description of the preferred embodiments presents a description of certain specific embodiments to assist in understanding the claims. However, the present invention can be embodied in a multitude of different ways as defined and covered by the claims. Throughout this document, the words user, caller and patient are used interchangeably. However, it will be understood that the caller may be acting as a proxy for the patient.

I. Introduction

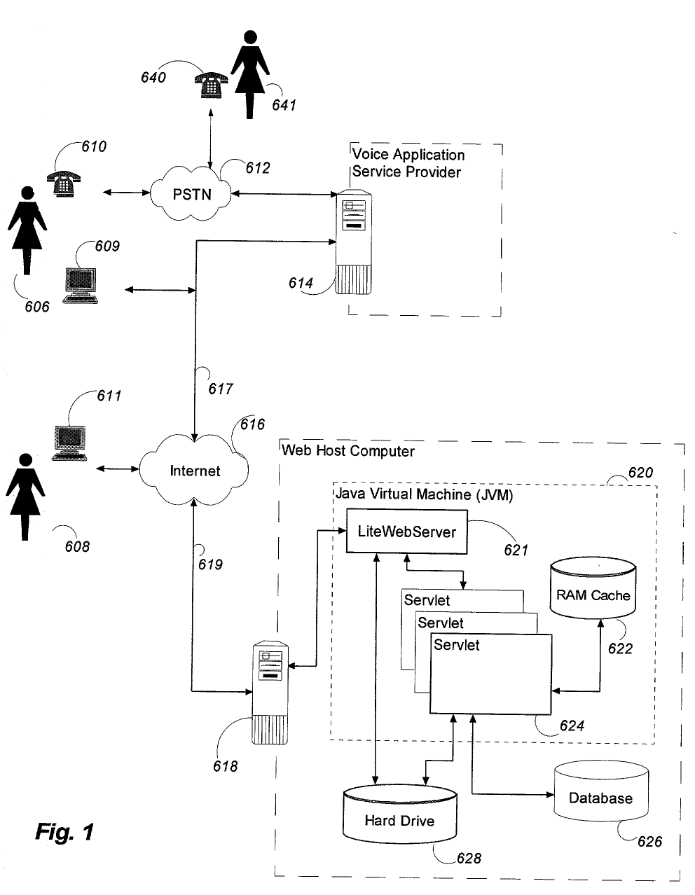

A consultation for a person seeking medical advice 641 begins with a telephone call to the medical triage (MT) system of the present invention. The MT system is currently available in two main modes of operation: a fully automated telephone triage (FATT) program, and a nurse-assisted telephone triage (NATT) system. The preferred embodiment of the present invention uses the nurse-assisted telephone triage system. The fully-automated telephone triage program is discussed in the Other Embodiments of the Present Invention section. Both the FATT and the NATT programs use the system configuration depicted in FIG. 1.

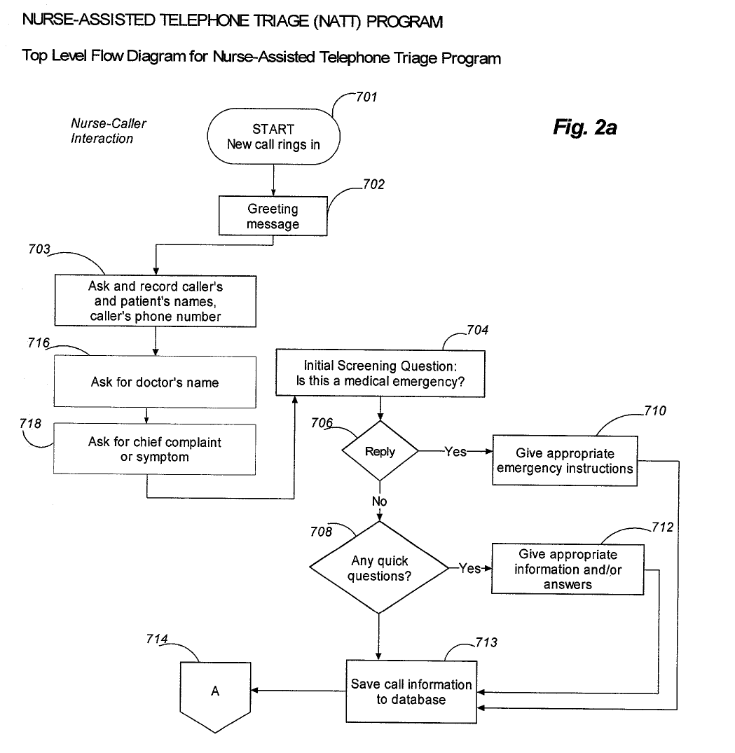

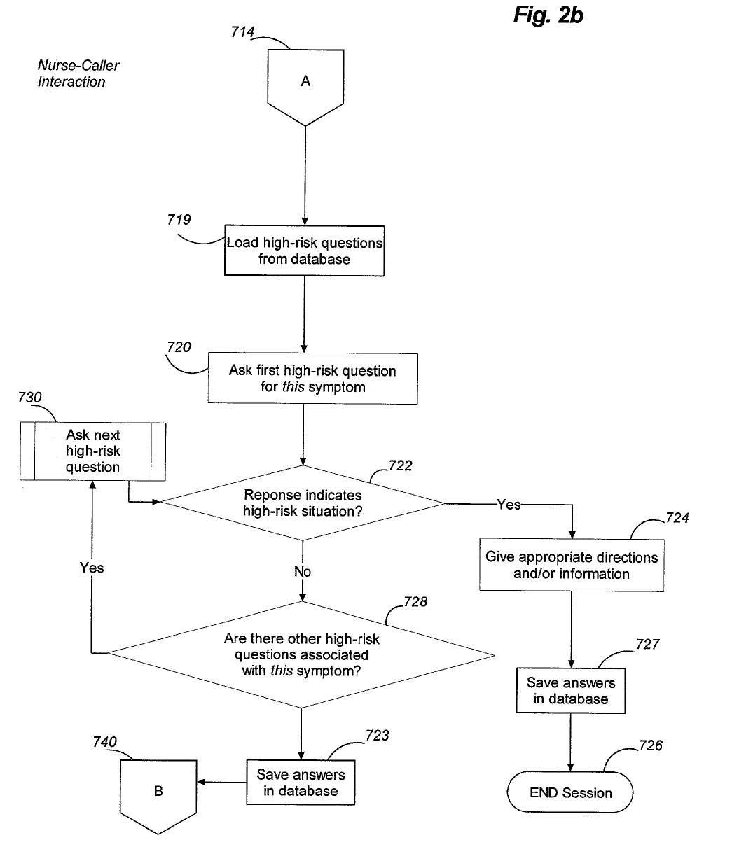

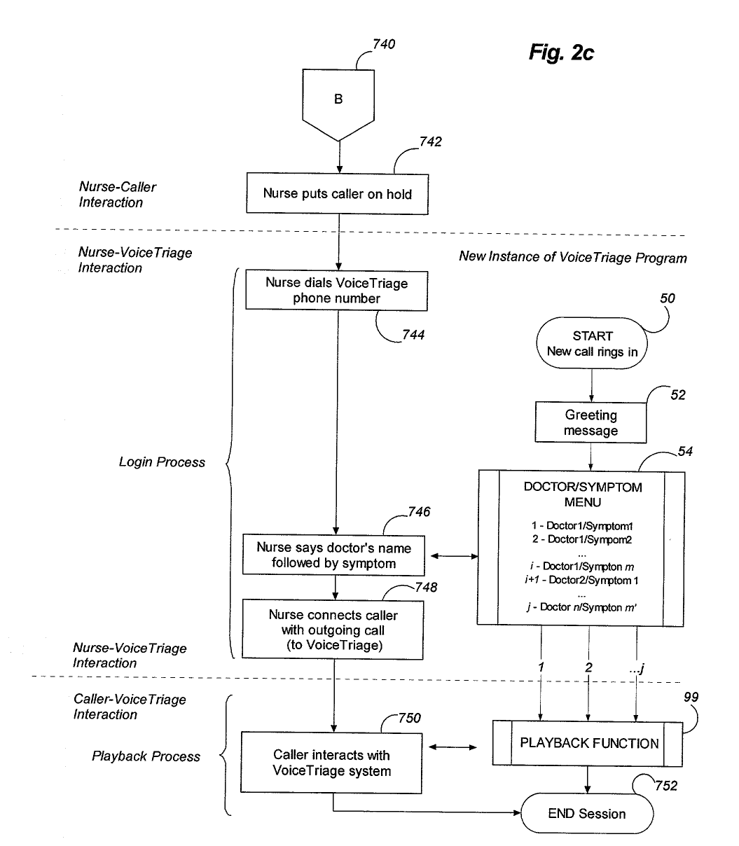

FIGS. 2a, 2b, and 2c are a top-level flow diagram for the NATT system. A person seeking medical advice 641 places a telephone call to a special telephone hotline. On the other end of the hotline is a medical nurse 606 that answers the call, asks a few medical questions, and then places the caller on hold. On another telephone line the nurse 606 dials and logs into the VoiceTriage program, the computer-automated portion of the NATT system. To log into the program, the nurse 606 must state the doctor's name and symptom after a particular computer prompt. There is a new instance of the VoiceTriage program for each incoming call. The nurse 606 logs into the VoiceTriage program, and connects the caller 641 to the VoiceTriage program just before an audio playback of a doctor's message begins. Both the caller 641 and the nurse 606 stay on the line until the VoiceTriage program has finished the message. Once the message is completed, all parties disconnect and the session ends.

II. System Overview

The hardware and system software were assembled with three basic concepts in mind: modularity in software design, portability to other operating systems, and the use of industry standard components. In this way, the system can be more flexible, allowing for additional embodiments of the invention. While specific hardware and software will be referenced, it will be understood that a wide array of different components could be used in the present system.

Referring to FIG. 1, the components of a preferred embodiment of the computerized medical triage system are shown. The voice application service provider 614 is a personal computer that is connected to a public switched telephone network (PSTN) 612 and a computer network such as the Internet 616. The voice application service provider (VASP) computer 614 handles the voice recognition, text-to-speech synthesis, and audio playback required by an interactive voice response (IVR) system. On a separate computer labeled as a web host 618, connected to the VASP computer 614 via a data network such as the Internet 616, resides a web server 619. The web host 618 includes a variety of components including program files on a hard drive 628, a Java Virtual Machine (JVM) 620, and a database 626.

The voice application service provider computer 614, the web host computer 618, and the nurses' computer workstation 609 currently are typical computer systems that include a processing unit to execute the instructions of the software; a display unit as a means of providing a computer user with the prompts and information necessary to practice the invention; an input device to provide the means for the computer user to interact with the software version of the invention; a storage device for storage of the software and the files associated with the invention; and an output device for printing reports and other information. VASP computer 614, web host computer 618, and nurse's workstation 609 include an Intel Pentium III microprocessor, a 20 gigabyte hard drive, and 320 megabytes of RAM. These three computers also have typical network connections that allow for high-speed access to the Internet 616. Such high speed connection is currently provided by a T1 line but other configurations are possible. A short list of alternate broadband connections include cable modem, ADSL, and satellite connections.

“Telephony” functions use Dialogic Corporation's D/240JCT-T1 voice processing (VP) board that communicates with the VASP computer 614 via the PCI bus. The voice processing (VP) board performs several functions including interfacing the telephone lines, decoding touch tone signals, and speech recording. Touch tone signals are also known as “dual tone multiple frequency” (DTMF) signals. The voice processing board connects to the public switch telephone network 612 via a T1 line. The VASP computer 614 may include a plurality of VP boards based on how many phone line connections are desired for the system.

Automatic speech recognition (ASR), text-to-speech (TTS) synthesis, and voice browsing is achieved using International Business Machines (IBM) Corporation's WebSphere VoiceServer product. The VoiceServer is a software product that operates with an existing Web infrastructure to allow delivery of voice applications. It uses industry standards such as VoiceXML (VXML), Java, and H.323, the Voice over IP (VoIP) standard. VoiceXML is a standards-based programming model for writing interactive voice applications. The system currently uses VoiceServer version 1.5 with Dialogic code as an add-on. VoiceServer includes the following features: U.S. English, U.K. English, French, and German language versions; a voice browser that interprets VoiceXML markup; IBM's award-winning speech recognition and Text-to-Speech engines; and scalable solutions using many industry standards such as VoiceXML, Java, and H.323 (Voice over IP standard).

In the presently preferred embodiment, the voice application service provider computer 614 operates under the Windows NT operating system. The TMT system software is written in VoiceXML 1.0 and in HyperText Markup Language (HTML) 4.0, both industry standards. VoiceServer runs on computer 614 under WindowsNT. The web host 619 operates under the Windows95 operating system. LiteWebServer (tm) version 2.2 of Gefion Software Co. is the web server currently being used. It requires a Java virtual machine to be present on the web host 619, currently Java SDK 1.2 of Sun Microsystems, Inc. Java servlets were created and executed using the Java(tm) Servlet Development Kit 2.0 of Sun Microsystems, Inc. The database 626 is located on the web host 618 and has been created using Microsoft Access 97. Connectivity between the Java virtual machine and the database is achieved using the JDBC-ODBC drivers included in the standard development kits described above, namely Java SDK 1.2.

The combination of the VoiceServer program and the Dialogic D/240JCT-T1 board results in powerful voice recognition capabilities. The system allows for both continuous and discrete speech vocabularies and grammars. For example, the system can understand multiple digit numbers, single-word answers such as “yes” or “no”, and customizable grammars such as a list of names or phrases.

III. Authoring Languages

The telephone medical triage system is coded in an industry-standard authoring language called VoiceXML (VXML). Programming in VoiceXML is similar to programming with HyperText Markup Language (HTML). Web browsers such as Netscape Navigator or Microsoft Internet Explorer read HTML files and display their contents on the screen. To transmit text in readable form, the Internet software begins with ASCII code and “marks it up” with special codes to indicate special text handling such as bold, underlining, colors and fonts, and so on. Programmers design a human-to-computer dialogue using VoiceXML by “marking up” computer prompts, menus, and responses. To encode that a particular sound file should be played to the caller, VoiceXML may encode it as follows:

<audio src=“hi.wav”> Welcome to the triage unit. </audio>

The text string “<audio src=“hi.wav”>” is a VXML tag that indicates a sound file “hi.wav” should be played. In the case that the indicated file was not found or some other error occurred, the text between the “<audio>” and “</audio>” tags should be synthesized into speech. In the example above, the ASCII character string “Welcome to the triage unit” would be converted to audio, more specifically, synthesized into speech. VoiceServer has the text-to-speech synthesis engine required for such operation. The Dialogic voice processing board plays back the synthesized audio to a caller via the interfaced phone lines.

VoiceXML contains other markup tags such as if, goto, and submit. The submit tag is particularly useful since information collected from the user can be stored within a VoiceXML script and submitted to another program for processing.

Java Servlet technology is used to dynamically generate VoiceXML files. We consider static VoiceXML files to be those that have been predefined, and most likely stored on a fixed medium such as a hard drive. On the other hand, dynamically generated VoiceXML files are created on-the-fly. In the preferred embodiment of the present invention, VoiceXML files are dynamically generated by a generate file function 99 defined in a Java servlet listed below.

The Java Servlet class files 624 reside on a web server 618 connected to a computer network such as the Internet 616. Unlike other server-side scripting languages, Java Servlets are run from within a Java Virtual Machine (JVM) 620. Servlets 624 do not use the same random access memory space as other programs on the web server 618. Instead, their memory resources are contained within the Java Virtual Machine 620, resulting in increased security, reliability, and scalability. Furthermore, since the Servlet class files 624 are encoded in JVM byte code, and not in native byte code, these files can be run on any computer hardware platform that supports a JVM. For example, our system could easily be run on a Sun Solaris Workstation, instead of an IBM PC-compatible computer, because a JVM exists for that machine.

The system software includes the following code modules which are represented as flow diagrams.

VoiceTriage.vxml—a VoiceXML file that manages a dialogue between a caller and the VoiceTriage system.

VoiceTriageServlet—a Java servlet that dynamically generates VoiceXML files depending on a caller's doctor's name and chief complaint or symptom. The generated file is parsed by VoiceServer and played-back to the caller.

answers.gsl—a speech grammar file that defines a grammar for use by the speech recognition component of VoiceServer. This file defines the responses “yes” and “no”.

doctors.gsl—a speech grammar file that lists the names of all the doctors to be recognized by the MT system. A phonetic spelling of a doctor's name can be used when a speech recognition engine has difficulty recognizing a particular name.

symptoms.gsl—a speech grammar file that lists the symptoms to be recognized by the MT system. A phonetic spelling of a symptom can be used when a speech recognition engine has difficulty recognizing a particular word.

VoiceTriageNurseLogin.html—a HTML file that includes a form for data entry. Calls to the TMT system are logged by the nurse 606 using this form.

VoiceTriageNurseLoginServlet—a Java servlet that accepts a completed form (from VoiceTriageNurseLogin.html) and saves its information to a database 626.

VoiceTriageQuestionaireServlet—a Java servlet that both transmits blank forms containing high-risk questions to a client computer, and accepts completed forms containing answers to the high-risk questions from a client computer. Incoming completed forms are saved to a database 626.

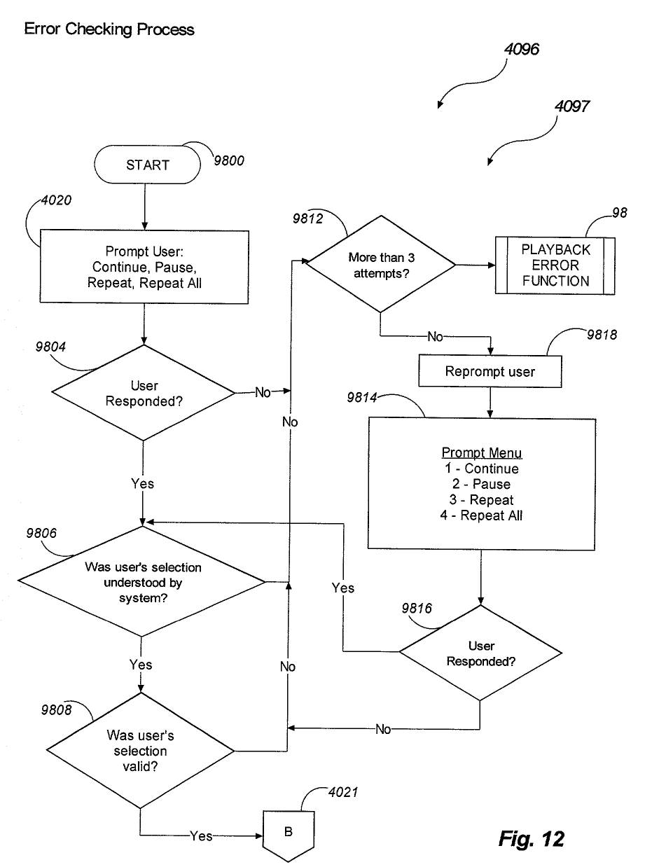

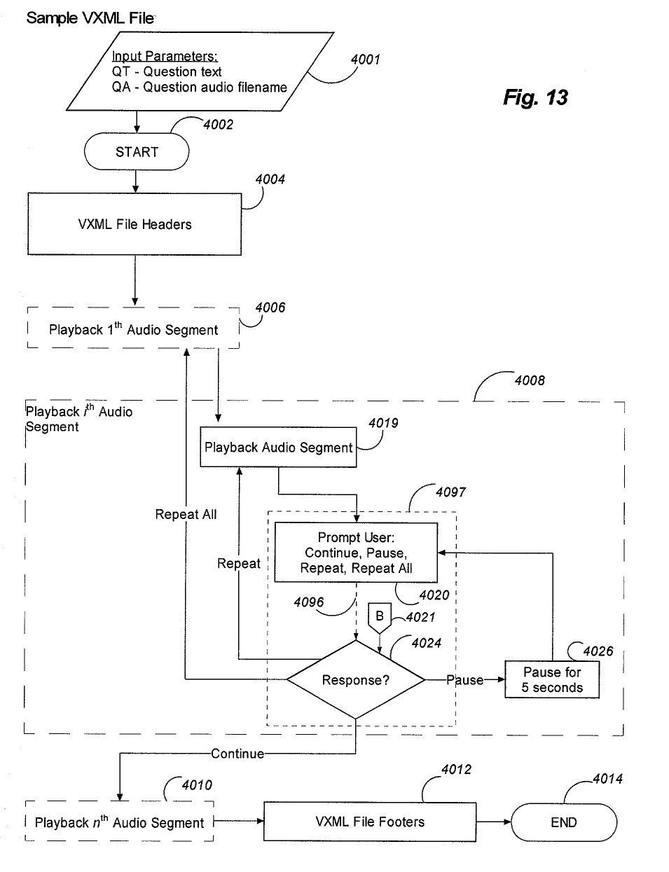

That there are relatively few code modules listed above is deceptive to the true size of the MT system. Suppose there are 20 doctors with an average of 10 symptoms each. That is, each doctor has his or her own advice for 10 common complaints. Then there are really 200 code modules (in addition to the ones listed above) since there must be a VoiceXML file for each combination of doctor and symptom. An example of a dynamically generated VoiceXML file is represented as a flow diagram in FIG. 12 and is discussed in the next section.

VoiceXML application files and speech grammar files are parsed by the VoiceServer program. Java Servlet source files are compiled by the Java compiler into Java bytecode using the standard Java 1.2 and Java Database Connectivity (JDBC) 2.0 class libraries.

IV. Run-Time Operation

A nurse-assisted telephone triage program session begins with a person requiring medical information 641 placing a telephone call from any device that is ultimately routed to a public switched telephone network 612 to the triage telephone hotline. The phone call can originate from a typical land-line telephone, a cellular, wireless or other mobile phone, or from a device connected to a Voice Over IP (VoIP) network that connects into the PSTN 612. The triage hotline is switched into the nurse's 606 telephone 610. Referring to FIG. 2a, the nurse 606 answers the incoming phone call 701, and recites a greeting message 702. Next, the nurse 606 asks for the patient's name, and the caller's name, in case they are not the same person (node 703). The nurse 606 then asks for the name of the patient's doctor (716). Afterwards, the nurse 606 asks what is the patient's chief complaint or symptom (720). The nurse 606 asks an initial screen question 704: “is this a medical emergency?” If it is, then the nurse 606 gives appropriate emergency instructions 710. The emergency instructions can range from first aid instructions to dispatching an emergency ambulance to the caller's location. If no medical emergency situation is present then the nurse 606 checks to see if the caller has any specific questions, indicated by node 708. These questions can range from requesting a doctor's appointment to clarification of doctor's orders. For example, worried parents may request more information about the appropriate dosage for fever reliever for their child.

The nurse 606 logs the telephone call interaction using a web browser such as Netscape Navigator on a personal computer 609. A form for data entry is found in the VoiceTriageNurseLogin.html file and its on-screen display is similar to FIG. 20. The nurse 606 enters the patient's name into field 2012, and the caller's name (if different) into field 2014. A nurse 606 selects the patient's doctor's name from a pop-up menu 2016, and the patient's chief complaint or symptom from a pop-up menu 2018. The nurse 606 selects her name from a pop-up menu 2020. If the call is an medical emergency the nurse 606 selects a “yes” toggle button 2022. Any and all comments are duly noted in the comments text box 2024. When the form has been completed, the nurse 606 clicks on a “Submit and Save Information” button 2026. Clicking on the submit button 2026 causes the web browser to transmit the form information to the voiceTriageNurseLoginServlet via a HTTP POST request. The VoiceTriageNurseLoginServlet Java servlet saves the fields of the form into their respective fields in the database table named NursesLogTable, shown in FIG. 18a.

Referring to FIG. 2a, after the nurse 606 asks the caller 641 for her name, her doctor's name, her chief complaint, an initial screen question, and for any quick medical questions, the nurse 606 saves the call information to a database 626 in the manner described above. If an incoming call had been a medical emergency, then the nurse 606 and the caller would disconnect if necessary.

If an incoming call is not a medical emergency then the VoiceTriage program transmits to the web browser a HTML page containing a list of high-risk questions. This HTML page originates from the web browser on computer 611 decodes the HTML file and displays its contents on the monitor screen. An example of a display of a decoded HTML file can be found in FIG. 20a. The nurse 606 then iterates through the list of questions and asks each one to the patient 641. An affirmative answer (“yes”) indicates a potentially high-risk question that requires immediate medical attention. The nurse 606 asks each question and logs the caller's responses on the HTML page by selecting either a “Yes” or “No” toggle item. The nurse 606 asks other diagnostic information such as the patient's temperature, if known. The nurse 606 records the caller's temperature in the “Comments” textbox near the bottom of the form.

If a high-risk situation exists, then the nurse 606 gives the caller 641 appropriate directions and/or information. These directions are found under a “Directions for High-Risk Situations” header of the HTML file. These directions are given by the patient's doctor and are intended to be followed under a high-risk situation for a given symptom. Doctor's directions typically include paging a doctor, visiting an hospital emergency room, or setting up a doctor's appointment for the following day. The nurse 606 submits and saves the call session information into the VoiceTriage database 626 by first clicking on the “Save and Submit Information” button at the bottom of the HTML web page. Clicking on the submit button 2026 causes the web browser to transmit the form information to a VoiceTriageQuestionaireServlet Java servlet via a HTTP POST request. Next, he servlet saves the fields of the form into their respective fields in a database table named HighRiskAnswerTable, shown in FIG. 18b. The session ends with all parties disconnecting and after the nurse has logged the call in the database.

If it has been determined that no high-risk situation exists, then the nurse places the patient's call on hold and dials the VoiceTriage phone number, as indicated in nodes 742 and 744 of FIG. 2c. The nurse 606 has access to multiple phone lines in her office location where a typical office telephone system exists. This phone system includes a telephone 610 that can handle multiple incoming and outgoing phone lines which are connected to a public switch telephone network 612.

The nurse's outgoing call to the VoiceTriage program is switched through the PSTN 612 to the voice application service provider computer 614. The Dialogic voice processing board on the VASP computer 614 picks up the incoming call and the VoiceServer program invokes a new instance of the VoiceTriage program, described in VoiceTriage.vxml. Referring to FIG. 2c, once the incoming call has been answered by the VoiceTriage program, a greeting message 52 is played.

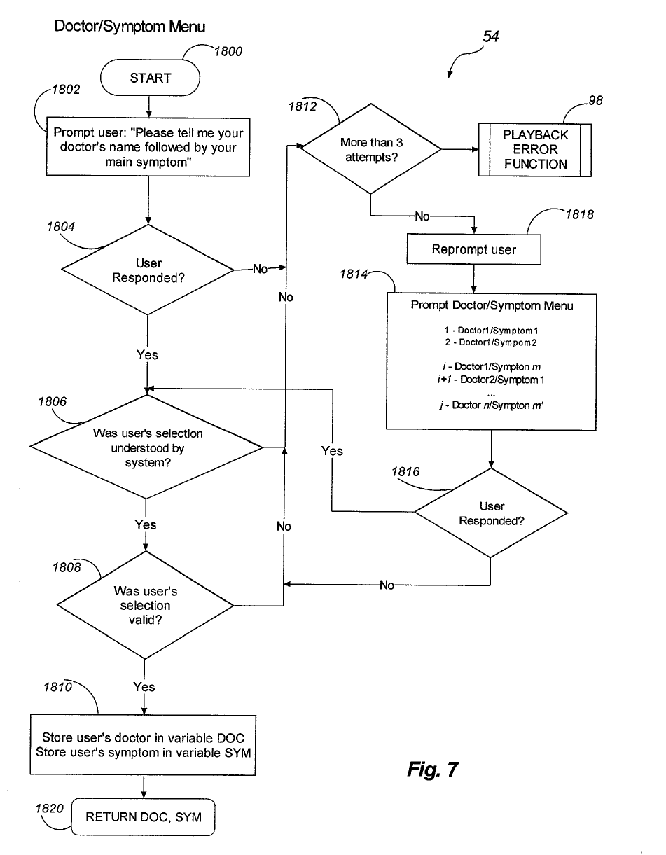

Next, the nurse 606 is presented with a Doctor-Symptom menu 54. As shown by node 1802 of FIG. 7, the VoiceTriage program first prompts the nurse 606 to enter their selection. The VoiceTriage program then waits for the nurse 606 to respond. If no response was heard after a specified amount of time, currently 5 seconds, then the system prompts the nurse 606 again (1818). Additional information regarding a list of possible selections is given to the nurse 606 in audio form in plain English. Multilingual audio playback is available with the VoiceTriage system and is described in more detail in the Other Embodiments of the Present Invention section.

At this point (after prompt 1802) the nurse 606 speaks the name of patient's doctor and the patient's symptom into the telephone. The automatic speech recognition (ASR) engine, part of the VoiceServer program, processes the nurse's speech into machine-intelligible form. Given that the nurse responded to the initial prompt, the next question is whether the nurse's selection was understood by the system (decision 1806). If not understood, then the VoiceTriage program indicates to the caller that their selection was not understood and to try again prompt 1818).

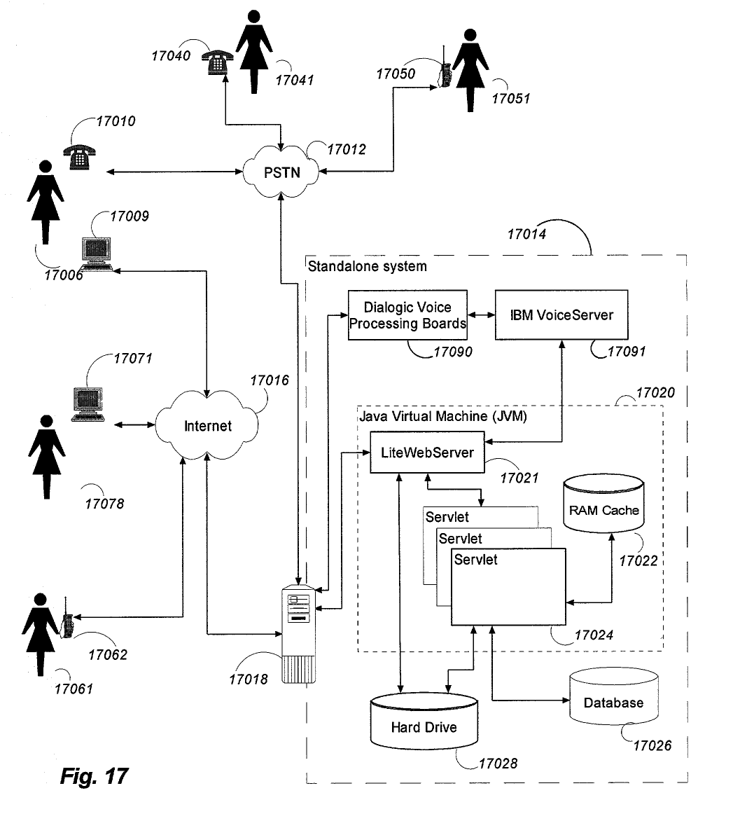

Assuming that the nurse's selection was understood by the system the VoiceTriage program checks to see if it is a valid selection (decision 1808). The architecture of the VoiceTriage program is such that the only words recognizable by the system at decision 1806 are also valid responses for decision 1808. In other words, the system only recognizes words or phrases that are valid selections for the doctor-symptom menu. This is because there is a specific grammar file associated with each menu. For example, the words “yes” or “no” that are understood and are valid in other menu contexts are not valid in the doctor-symptom menu 1814 (and 1802). VoiceXML allows for the specification of different grammars and vocabularies whenever the user is prompted to speak. The VoiceTriage program allows for three attempts for each of the following checkpoints: “user responded?” 1804; “user selection understood by system?” 1806; and “user's selection valid?” 1808. In the unlikely event that there was an error after three attempts, the system plays a general error message as defined by the playback error function 98 in FIG. 17. If the user's selection is valid (decision 1808), then the system confirms what it thinks it heard with the user. The VoiceTriage program asks the nurse 606, “I heard you say DOC and SYM. Is this correct?” The name of the doctor and symptom understood by the system is replaced for the placeholders DOC and SYM, respectively. The nurse 606 responds with a “yes” or a “no”, or their DTMF equivalents, “1” or “2”.

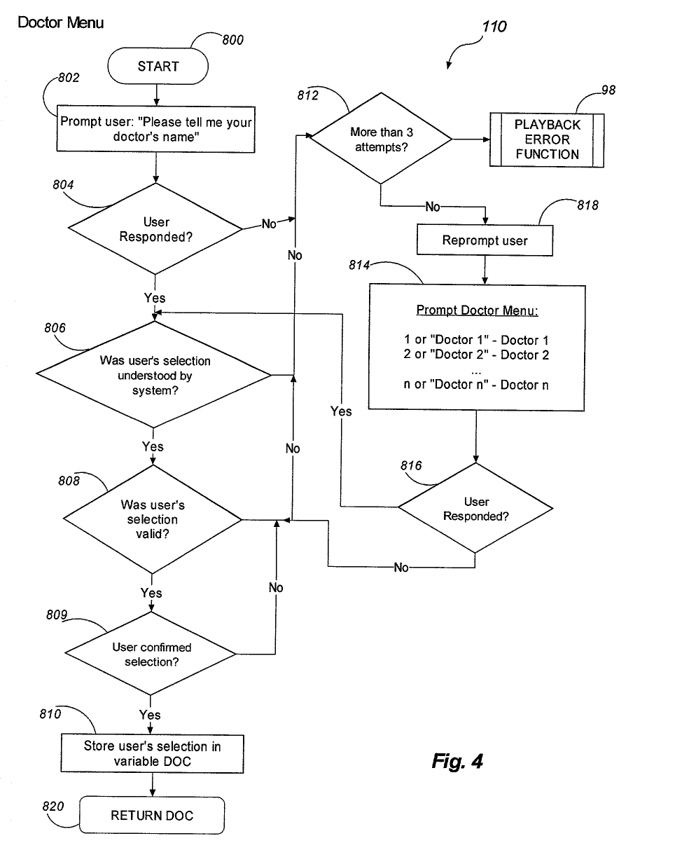

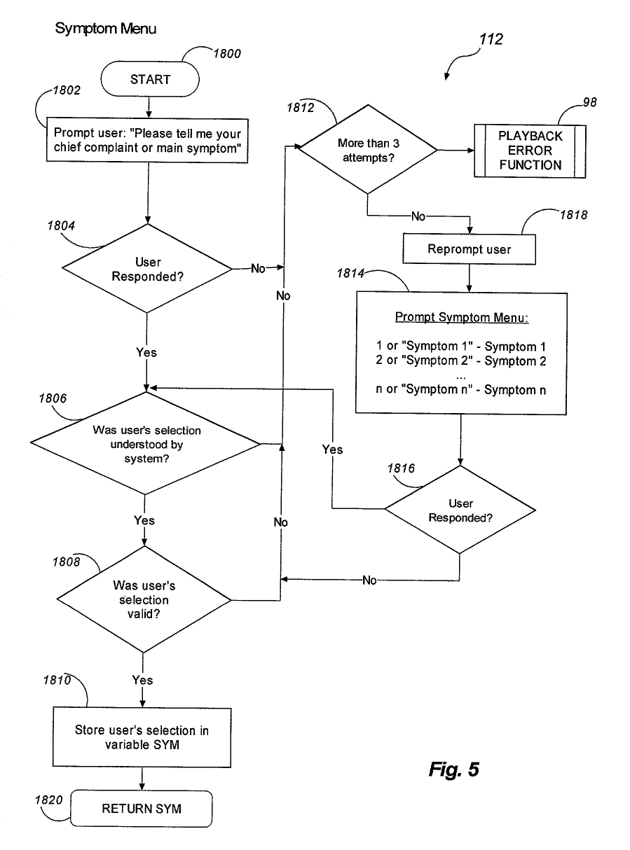

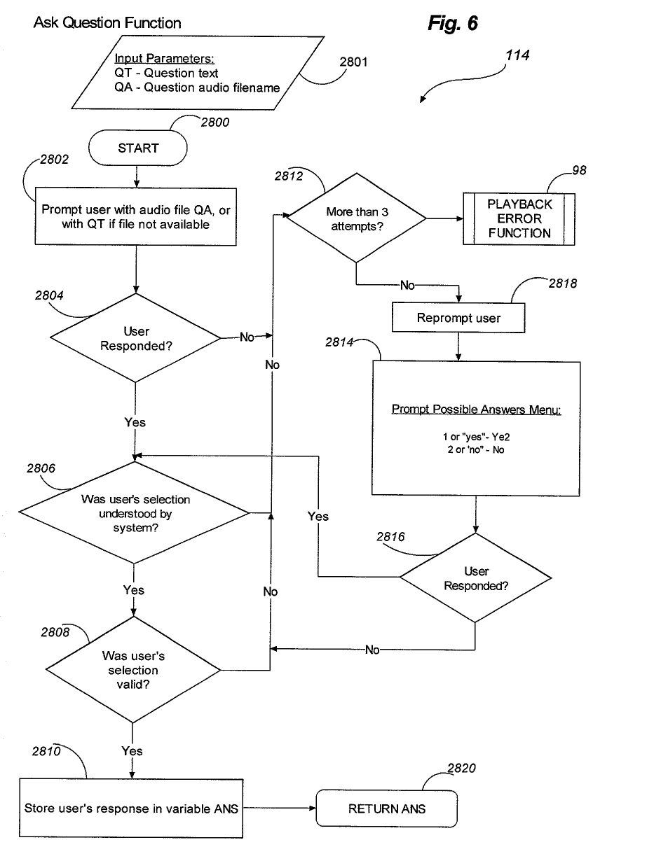

The general framework of checking to see if a user responded to a prompt, and checking to see whether the selection was understood and then confirm what was heard, can be thought of as a general error-checking process. These processes have been made explicit in the doctor menu 110, symptom menu 112, ask-question function 114, and doctor-symptom menu 54 flow diagrams (FIGS. 4, 5, 6, 7, respectively). However, it is noted that similar error-checking processes occur whenever a user is prompted to make a selection or enter information, and that these processes have been made implicit in some flow diagrams for the sake of clarity.

Once a selection has been confirmed with the user, the system stores the patient's doctor's name and symptom in variables DOC and SYM, respectively, as indicated in block 1810. These variables are now accessible to other parts of the VoiceTriage program.

Returning to FIG. 2c, once the nurse confirms her selection with the VoiceTriage program, she takes the incoming call off hold, and connects it with the outgoing call to the VoiceTriage program (node 748). Thus there is a three-way conference call between the incoming caller 641, the nurse 601, and the call to the VoiceTriage program.

The caller 641 now is the main person interacting with the VoiceTriage system during the playback process 99. The VoiceServer program plays the first audio segment to the caller 641. The audio segment includes medical instructions and advice in the patient's own doctor's voice for the caller 641 to follow. A typical call goes as follows: the first audio segment includes a greeting from the doctor as well as a brief review of criteria that would indicate a high-risk condition. The caller 641 is then asked if she wants to continue, pause playback for a moment, repeat the current audio segment, or begin audio playback from the first audio segment. Most callers will choose to continue with the next audio segment which typically includes medical instructions and advice. Certain symptoms may need longer or shorter medical instructions or messages from the doctor, so the number of audio segments varies with each symptom and doctor. Once all audio segments have been played, the caller 641 has the option of talking with the nurse 606 again, or to end the session and disconnect. Naturally, the caller 641 has the option of disconnecting and dialing the nurse 606 directly to ask additional questions.

1. Playback Function in Detail

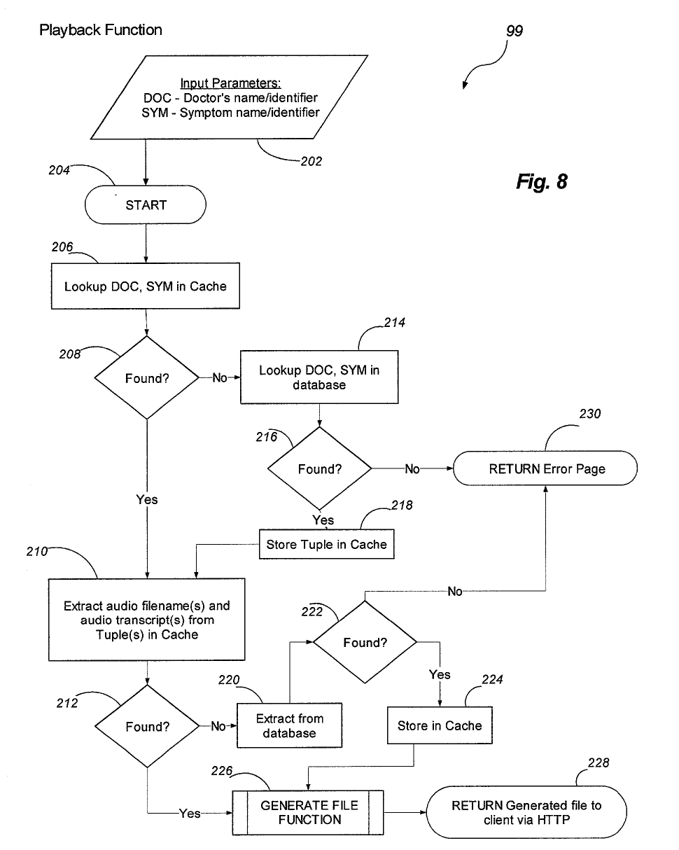

This section describes in more detail the process in which a playback function 99 (FIG. 8) dynamically generates VoiceXML files depending on the values of its input parameters.

A Playback function 99 is called after a user has logged into the VoiceTriage system. For the nurse-assisted telephone triage system, the function 99 is called after a nurse has selected a doctor and a symptom from the Doctor-Symptom menu 54. The function 99 is called from within the VoiceTriage.vxml VoiceXML file using the submit tag as follows:

<submit next=“http://mywebhost.com/servlet/VoiceTriageServlet” method=“post” namelist=“patientName DOC SYM”/>

(Note: “mywebhost.com” would have to be replaced by actual the domain name or IP address of the web host computer 618.) Referring to FIG. 8 and the above example, a doctor's name and a symptom name is passed into the function 99 as input parameters DOC and SYM, respectively. DOC and SYM are unique numerical identifiers and not the actual doctor's names. By using unique numerical identifiers we avoid potential confusion that could result if there existed two doctors with the same name.

The playback function 99 is a component of the VoiceTriageServlet Java servlet, which returns a dynamically generated VoiceXML file. A new instance of the VoiceTriageServlet servlet is instantiated for each new call to the playback function 99 shown in the flow diagram. The playback function 99 first searches for DOC and SYM in the cache 622. If not found, then the function 99 searches for records containing the values for DOC and SYM in the database 626, specifically table called DocSymTable table 2102 of FIG. 21. If records containing the values of DOC and SYM are not found, then a VoiceXML file with an error message is returned (block 230). Records matching DOC and SYM are otherwise stored in the cache 622 (block 218).

FIG. 21 lists the key tables of the VoiceTriage database 626. A table labeled “DocSymTable” 18010 holds a unique object identifier, a doctor's name, a symptom, a reference to a set of audio transcripts (ATSs), and a reference to a set of audio filenames (AFNs). A database table named “AFNTable” 18030 stores a unique object identifier, a reference number, an order-index value, and the file path and file name for a audio file. A database table labeled “ATSTable” 18020 contains a unique object identifier, a reference number, a order-index value, and a transcript of an audio file encoded as ASCII text. It is understood that the administrator of the VoiceTriage database 626 will ensure that there is exactly one audio transcript for every audio file, and that the order-index value for an audio transcript matches the corresponding order-index value for an audio file. This guarantees that the audio transcript matches the audio file to be played.

After the VoiceTriage program searches the database 626 for a record containing user-specified values of DOC and SYM, the record is stored in a random-access memory cache 622. There should be only one record entry per doctor-symptom combination in table DocSymTable 2102 of FIG. 21. This ensures that a given doctor has only one message for a given symptom. Next, the VoiceTriage program searches for all audio filenames and all audio transcripts referenced by the doctor-symptom record in the cache 622 (block 210). If no records are found, then the application searches in the database 626 (block 220). As before, all records matching the search criteria are stored in the cache 622. If no records are found or if an error occurs, then an error page is returned 230 and the session ends.

The audio transcripts and audio file names from the ATSTable 18020 and AFNTable 18030 records, respectively, are stored in two separate arrays and passed as input parameters into a generate file function 226.

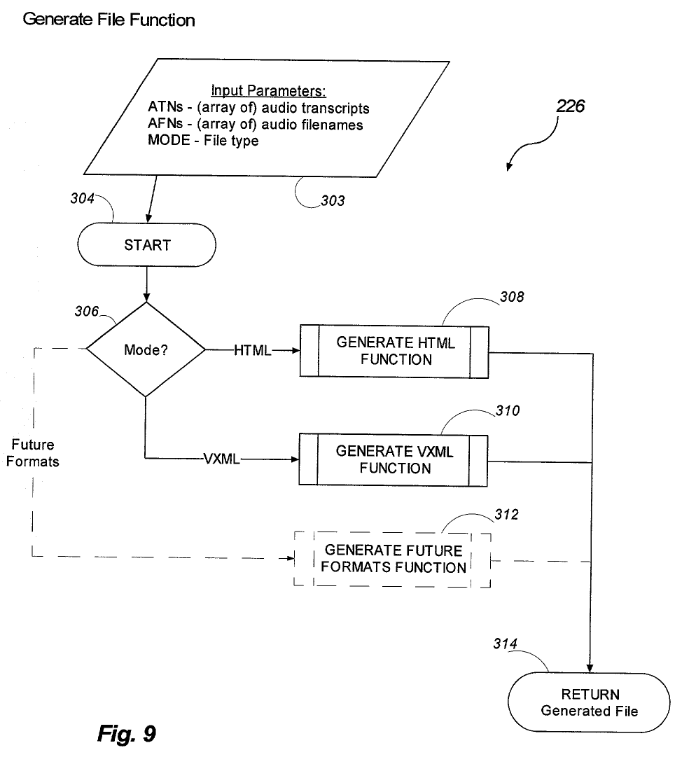

Referring to FIG. 9, the generate file function 226 also accepts a MODE as an input parameter in addition to ATSs and AFNs, arrays of audio transcripts and audio file names, respectively. This feature of the preferred embodiment of the current invention is an example of modular code design. Additional modes of operation can be defined without significantly changing the original invention. For example, at decision 306, if MODE equals “HTML” then a generate HTML function 308 is called. If MODE equals ° “VXML” then a generate VXML function 310 is called. For calls placed to the VoiceTriage program the default mode will be “VXML” since VoiceXML files allow for telephony functions and communication. Other file formats are possible, as indicated by a generate future-formats function 312 in FIG. 9. Applications of additional file formats can include but are not limited to web-enabled wireless phone displays, and hand-held computing devices. Thus the only necessary change in the original invention to support additional file formats is the addition of a new MODE to decision 306, and associated function(s) to handle the new file format. HTML and other additional file formats are discussed in the Other Embodiments of the Present Invention section below.

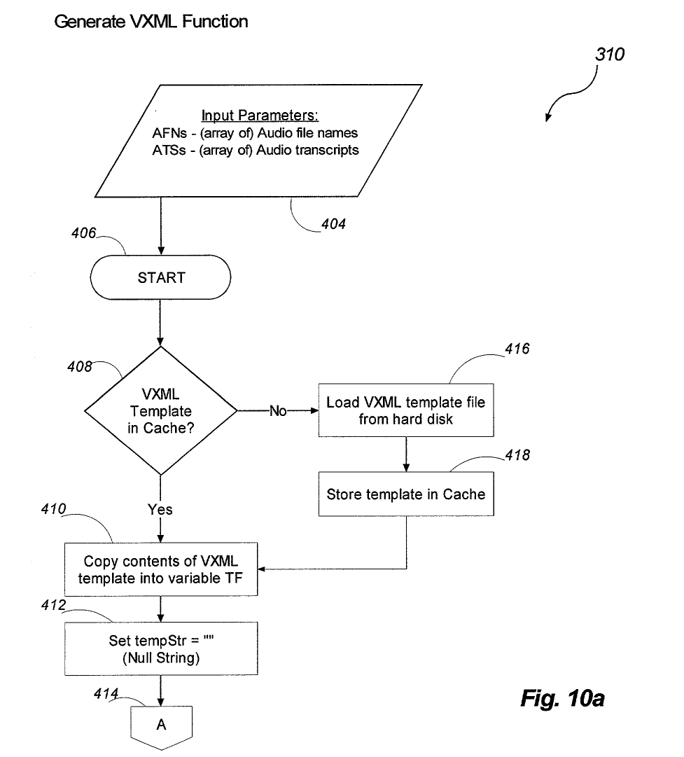

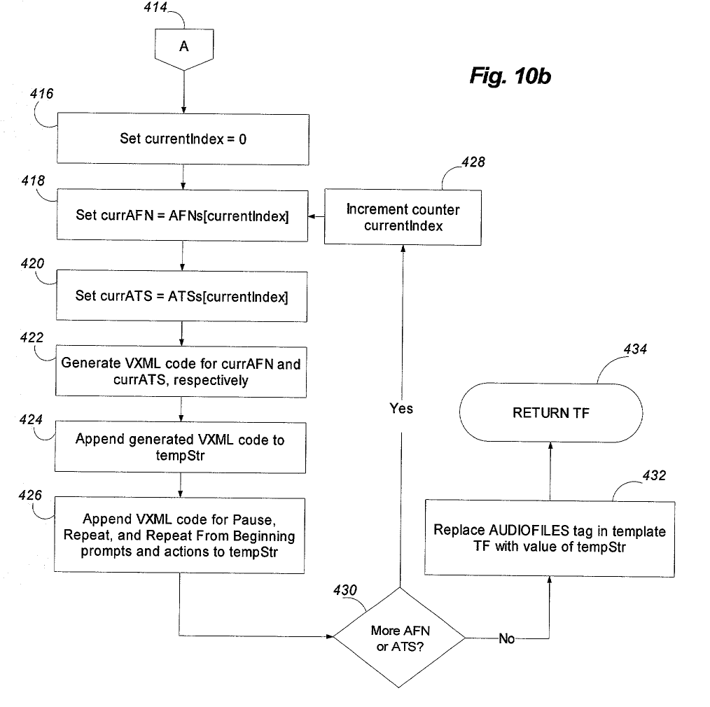

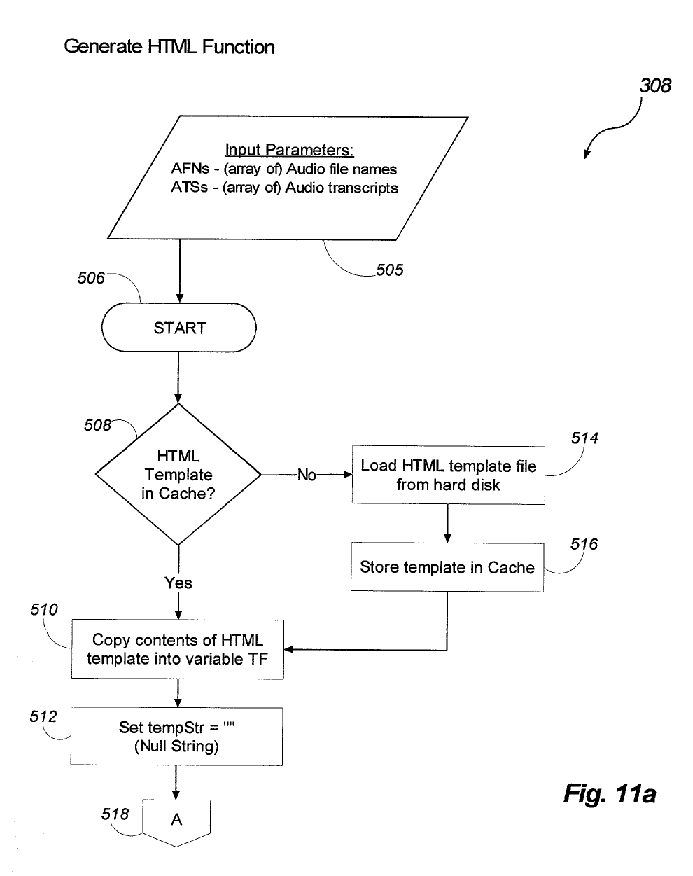

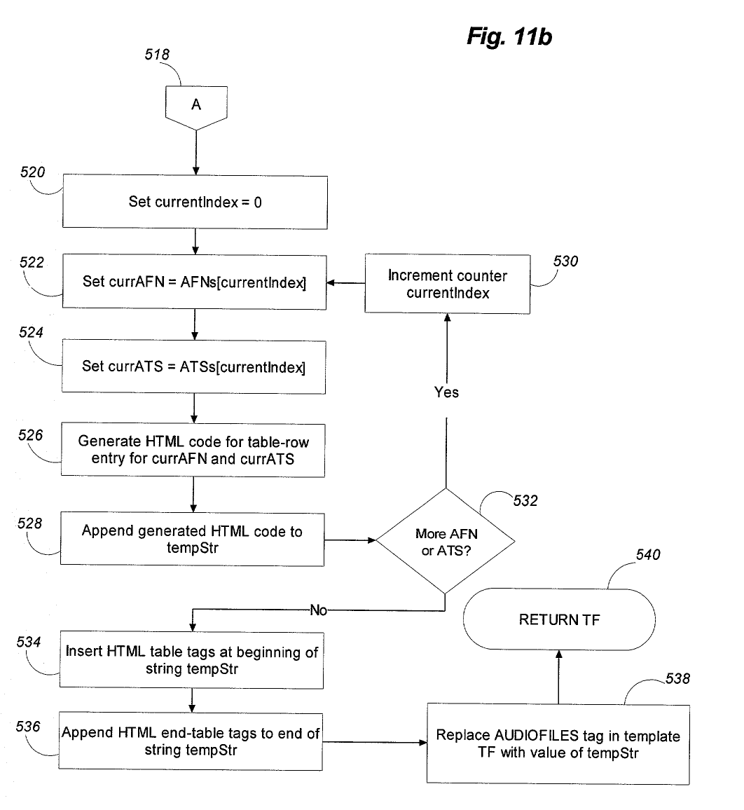

FIGS. 10a and 10b are a flow diagram of the generate VXML function 310. The function 310 expects two input parameters: AFNs, an array of audio fine names; and ATSs, an array of audio transcripts. First, the generate VXML function 310 checks to see if a template file exists in the cache 622 (decision 408). If not, then the template file is loaded from a hard disk and stored in the cache 622. The VMXL template file is duplicated and stored in a variable TF. Variable TF will ultimately contain a dynamically generated VXML file. A temporary variable tempStr is initialized to the null string (also called the empty string).

Next, the function 310 enters a loop that cycles through all the elements of the AFNs and ATSs arrays. A local counter currentIndex is initialized to 0. The main body of code for the loop (blocks 418, 420, 422, 424, and 426) is as follows: set variable currAFN to the value of array AFNs at array index currentIndex; set variable currATS to the value of array ATSs at array index currentIndex; generate VXML tags and code for currATS and currAFN; append the generated VXML code to variable tempStr; and finally, append VXML code for Pause, Repeat, and Repeat From Beginning prompts and actions to variable tempStr. If there are unprocessed elements left in the AFNs or ATSs arrays, then Function 310 increments the counter currentIndex and loops back to node 418.

Variable tempStr may look something like the following:

<audio src=“AFN1.wav”>This is a transcript of audio file AFN1.wav. </audio> <!-- This is a VXML comment indicating that the Pause, Repeat, RepeatAll, and Continue prompts go here. -->

<audio src=“AFN2.wav”>This is a transcript of audio file AFN2.wav. </audio> <!-- This is a VXML comment indicating that the Pause, Repeat, RepeatAll, and Continue prompts go here. -->

<audio src=“AFN3.wav”>This is a transcript of audio file AFN3.wav. </audio> <!-- This is a VXML comment indicating that the Pause, Repeat, RepeatAll, and Continue prompts go here. -->

The VoiceXML code for the pause, repeat, repeat all, and continue prompts have been omitted in the example above for sake of brevity. The example above is meant to show that a distinct set of audio tags and prompts are used for each audio file name and audio transcript pair. As seen above, the audio file name is inserted between quotes after the equals sign in the “<audio arc=>” tag; and the audio transcript is inserted between the “<audio src=“filename”>” and “</audio>” tags. The audio file name and audio transcript text differs for each doctor-symptom combination. Since this code segment is generated during run-time operation, we call the resulting VoiceXML file a dynamically generated file. Note that the audio file need not be physically located on the voice application service provider computer hard drive. The “audio file name” can be a uniform resource locator (RL). URLs specify the location of files in the world-wide-web. In this manner the audio file can reside anywhere in a computer network, as long as the URL can point to that file location. In the following example, “<audio src=“http://mywebhost.com/myAudioFile.wav”>”, the VoiceServer program would attempt to load and playback a file called “myAudioFile.wav” from the web server at “mywebhost.com”.

Once all audio segments have been processed, then the function 310 replaces a placeholder tag labeled “AUDIOFILES” found within the variable TF with the value of the string variable tempStr. The function 310 returns the contents of string variable TF, which has been shown to be a dynamically generated VXML file.

Referring to node 314 of FIG. 9, the generate file function 226 in-turn relays the generated file back to the playback function 99. In FIG. 8, the playback function 99 transmits the generated file to the client computer via HTTP. The playback function 99, generate file function 226, and generate VXML file function 310 all reside in the VoiceTriageServlet Java servlet on the web host computer 618. The main VoiceTriage.vxml VoiceXML application program had invoked the VoiceTriageServlet via a VoiceXML submit tag. Now that the execution of the VoiceTriage servlet has ceased, the dynamically generated VXML file is transmitted back to the VASP computer 614 for VoiceServer to decode and parse. The generated VXML file is encoded in ASCII and includes standard VXML markup tags.

VoiceServer now guides the caller through the dynamically generated dialogue. A flow diagram of an example of a dynamically generated VXML file is found in FIG. 12. A file begins with the necessary and required VXML file headers. Next, for explanatory purposes, the “body” of the VXML file is divided into n audio segments. We define an audio segment to include playback of an audio file; prompting the user if they want to continue, pause, or repeat audio segments; and any error checking involved with the prompts. However, this is just a semantic description of the file. In the actual VXML file only standard VoiceXML tags are used. FIG. 12 shows three audio segments: Playback 1^{th} Audio Segment 4006, Playback i^{th} Audio Segment 4008, and Playback n^{th} Audio Segment 4010. All three Segments follow the same flow diagram as indicated within Segment 4008, which is meant to be illustrative of the audio playback process. The total number of audio segments in the VXML file depends on the number of array elements in ATSs and AFNs variables of the playback function 99. Finally, the file ends with a VXML file footer.

X—Other Embodiments of the Invention

1. Fully-Automated Telephone Medical Triage System

A fully-automated telephone triage program follows a very similar telephone protocol as the nurse-assisted telephone triage system. Instead of communicating with a nurse 606, callers 641 talk to an computer program. We refer to the fully-automated telephone triage program as the FA-VoiceTriage program in the test of this document.

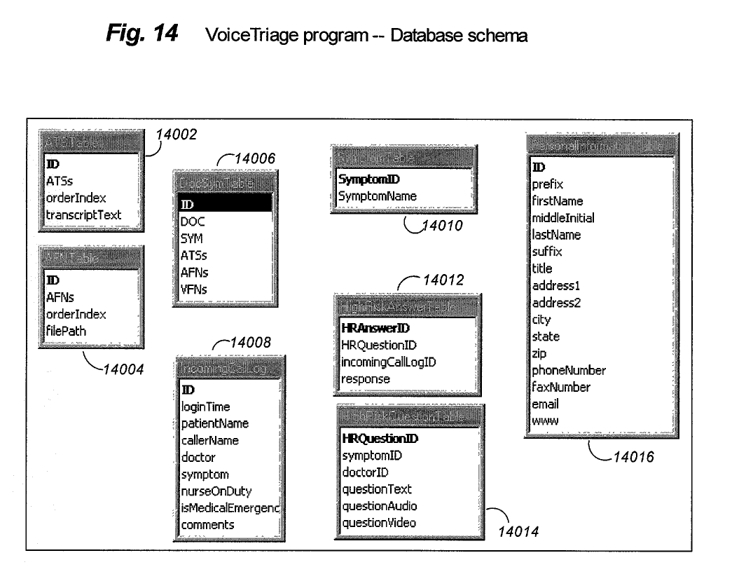

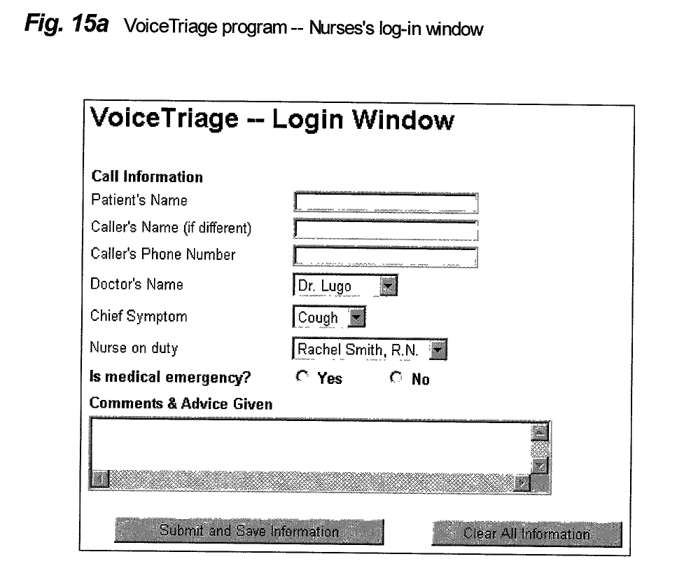

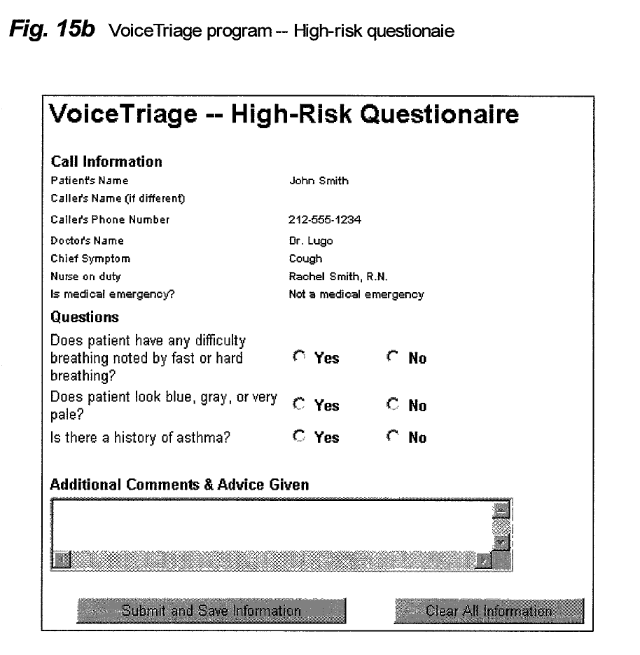

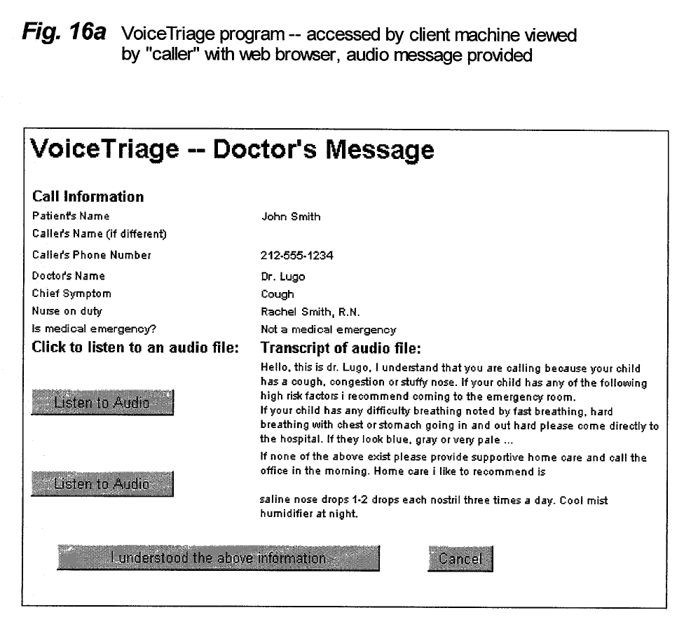

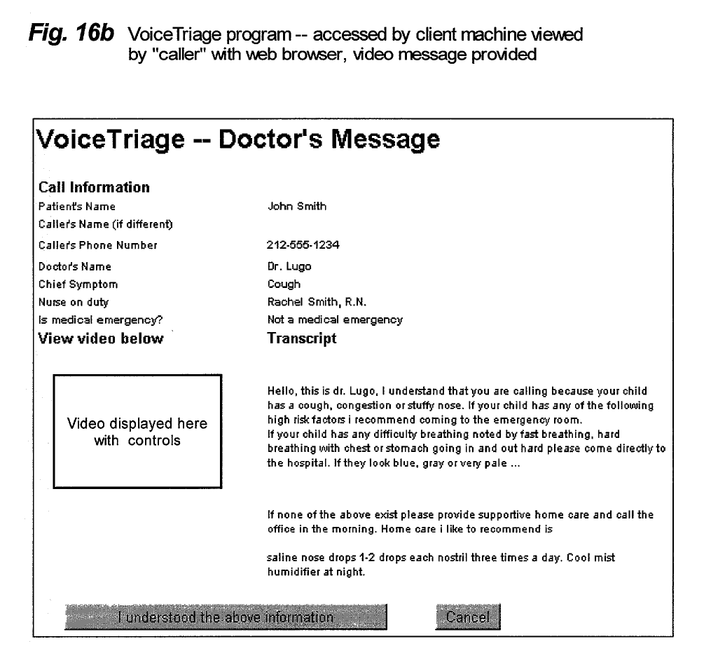

Easy access to the information in the FA-VoiceTriage system is made possible by a natural user interface. The computer-driven dialogue consists of simple yes/no questions, multiple choice questions, and questions requiring numerical values, such as the patient's body temperature. Voice recognition and interactive voice response technology allow callers to respond to yes/no questions, multiple choice questions, and questions required a numeric answer either by speaking directly into the telephone or by using the touch tone pad of their telephone. The questions and treatment recommendations are very simply worded yet skillfully designed to reflect the accumulated experience of many physicians in conducting patient interviews. The FA-VoiceTriage system is customizable and allows for the personalization of the computer-driven dialogue. For example, like the nurse-assisted VoiceTriage system, doctors can add their own high-risk questions to the standard computer-driven dialogue if they wish. This customization is performed by the database administrator who adds record entries in the appropriate tables, listed in FIG. 14, of the VoiceTriage database 626. Furthermore, the system can understand and respond to other languages besides English, such as French and German. The automatic speech recognition engine provided by IBM's VoiceServer can recognize a multitude of languages, and the text-to-speech engine of the VoiceServer product can similarly synthesize text into the speech of a multitude of languages.

Although all the TMT system's questions are designed to be easily understood, unforeseen situations will inevitably arise. For this reason, the caller always has the option of being reconnected to a live nurse 606, as shown in FIG. 1. Registered nurse 606 is affiliated with a doctor's office, medical answering service, or a local hospital. The registered nurse 696 has access to the VoiceTriage system either via a conventional personal computer 611 connected to the Internet 616, or through the use of a local, stand-alone installation of the VoiceTriage system on the local computer 611.

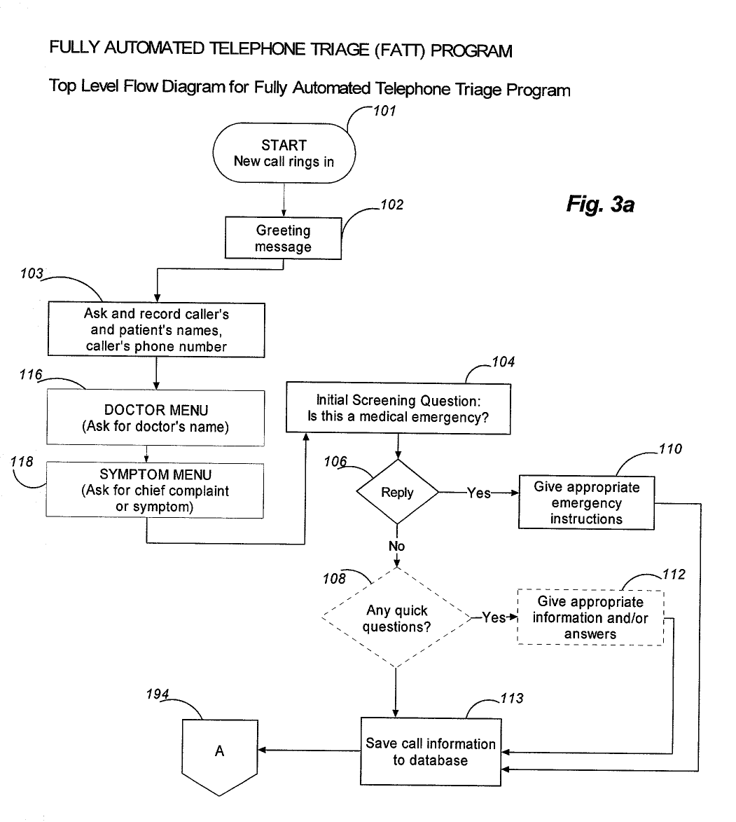

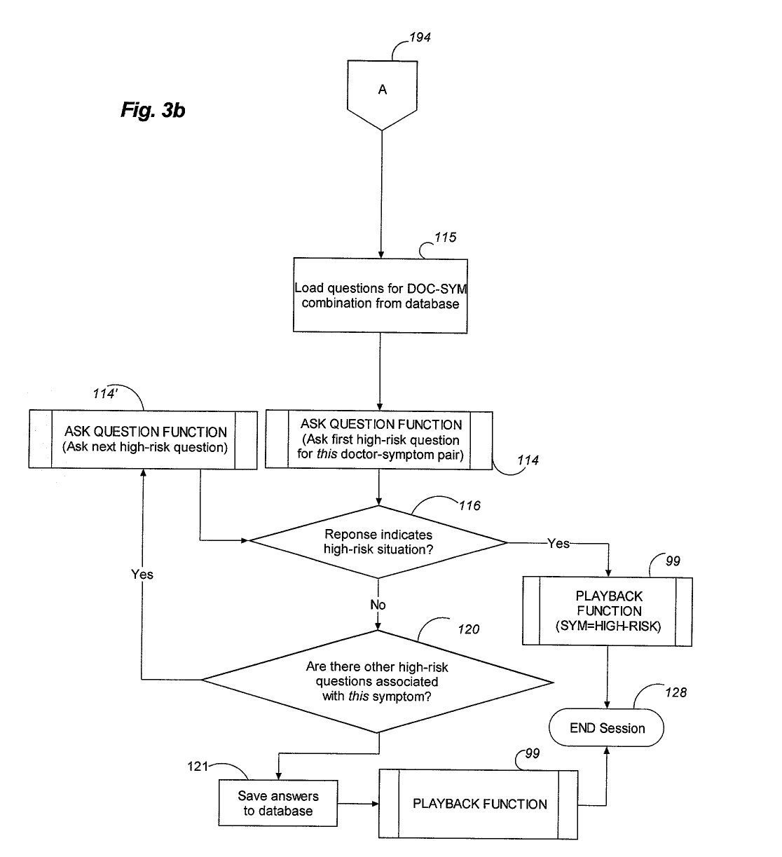

FIGS. 3a and 3b are a top-level flow diagram of the FA-VoiceTriage system. In addition to the code modules listed for the preferred embodiment of the present invention, the following code modules are used for the FA-VoiceTriage system:

FA_VoiceTriage.vxml—a VoiceXML file that manages a dialogue between a caller and the FA-VoiceTriage system.

FA-VoiceTriageLogin.html—a HTML file that includes a form for data entry. Provides a means for accessing the FATT program by a means other than voice/telecommunications, in this case, through a data network using a web browser that allows for browsing of HTML files.

FA-VoiceTriageIncomingCallLoginServlet—a Java servlet that accepts a completed form (from FA-VoiceTriageLogin.html) and saves its information to a database 626. Returns a questionnaire form to determine a high-risk condition in a file format depending on the MODE of the system: for voice applications a VXML file is returned, for web applications a HTML file is returned. Incoming information contained in aforementioned form is stored in a database 626. The servlet can handle a variety of forms, including but not limited to VXML, and HTML.

FIGS. 3a and 3b show a top-level flow diagram for the FATT program. Upon inspection it is clear that the flow diagram is very similar to a top-level flow diagram of the NATT program, the preferred embodiment of the present invention. The key difference is that a caller is always communicating with the computerized system in the FATT program, whereas in the NATT program the dialogue switch from caller-nurse, to nurse-VoiceTriage, to caller-VoiceTriage. Furthermore, the fully automated systems allows for a plurality of user interfaces, including but not limited to a speech interface by means of a telecommunications network, a graphical or text user interface by means of a data network with a web browser or similar device for the display of said interface, and other interfaces such as a display on a web-enabled mobile phone.

Conclusion, Ramifications, and Scope of Invention

Thus the reader will see that the method of the invention provides a reliable, consistent,

While my above description contains many specificities, these should not be construed as limitations on the scope of the invention, but rather an exemplification of one of preferred embodiment thereof. Many other variations are possible. For example, a stand-alone computer system wherein the functions and components of the voice application service provider computer, the web host computer, and the nurses' client computer all reside within the same physical computer system. Other combinations thereof, in which the functions and devices of the previously mentioned computer systems are arranged in different combinations depending on an application need. Other variations of the number of voice processing boards, and the manufacturers of such voice processing boards may differ, also, as long as a combination of other voice processing boards and audio processing boards lead to the same useful result, that being a means for the following items: processing audio, processing speech, performing voice recognition, synthesizing text into speech, and playback of audio. A medical triage system in which callers call using a mobile phone, a wireless phone, a CB radio, a radio communications device, a satellite telephone, satellite communications device, a hand-held computer, a portable computer, a computer system connected to a wireless network, computer systems connected to intranets, the Internet, extranets, and other typical types of computer networks. Variations in languages understood by a computer system, including but not limited to English, French, German, Spanish, Chinese, and other Asian languages. Other variations in the hardware configuration of the voice application service provider computer such as using other third-party vendors including TellMe Networks, Nuance Systems.

Accordingly, the scope of the invention should be determined not by the embodiments illustrated, but by the appended claims and their legal equivalents.

We claim:

1. A method of providing medical triage information over a communications network, the method comprising:
a person seeking medical advice;
a medical practitioner;
a telecommunications network;
a data network;
a computer system comprising
a display device providing the means for a user to view prompts and information necessary to practice the current invention;
an input device providing the means for the said user to interact with the software component of the invention
a processing unit for manipulation of data and for execution of software;
a storage device for storage of the software and the files associated with the invention;
a device for interfacing with said data network;
said data network interconnecting a plurality of said computer systems
an algorithm for determining an appropriate medical response for a given symptom and a plurality of high-risk factors;
said person communicating via said telecommunications network with said medical practitioner;
said medical practitioner following said algorithm so that an appropriate response for said person's symptom is determined;
a means for providing medical advice to said person;
whereby said person receives medical advice from said medical practitioner via a telecommunications network and the determination of said medical advice is facilitated by execution of said algorithm.

2. The method of claim 1 wherein said telecommunications network comprising a plurality of devices including but not limited to a telephone, and a public switched telephone network.

3. The method of claim 2 wherein said telecommunications network further including a PBX telephone system.

4. The method of claim 1 wherein said telecommunications network further including a Voice over IP network.

5. The method of claim 1 wherein said data network comprises the Internet.

6. The method of claim 1 wherein said data network comprises an intranet.

7. The method of claim 1 further including:
software automating a portion of said algorithm;
software asking a user to log call information including but not limited to a patient's name, a caller's name, a phone number, a doctor's name, and a chief complaint;
software assessing a patient's level of risk;
a means for saving said call information, the patient's level of risk, and determined medical response to a storage device.

8. The method of claim 7 further comprising a transfer of information via said data network whereby storage of said information may occur across a plurality of remote computers in addition to the local computer.

9. The method of claim 1 wherein said means for providing medical advice to said person further includes said medical practitioner communicating to said person determined medical advice.

10. The method of claim 1 wherein said computer system further includes:
a first device for interfacing with a telecommunications system;
a first means for performing automatic speech recognition on an audio signal from first device;
a second means for synthesizing computer text into speech;
a user communicating with said computer system using spoken language by means of said first device and said first means;
said computer system communicating with and responding to said user using a natural language by use of said second means;
a plurality of audio files accessible by said computer system from a location selected from the group consisting of a local storage device, and a remote storage device located on a remote computer system accessible via said data network;
a third means for playback of said plurality of audio files via said first device;
a fourth means for prompting a user, asking said user a series of questions, selecting an appropriate audio file to playback based on said user's answers to said series of questions, and playing of the selected audio file via said telecommunications network using said third means thereby partially automating the means for providing medical advice to said person;
whereby the audio file played to said user via said telecommunications network contains medical information specific to said user.

11. The method of claim 10 wherein said natural language includes but is not limited to English, French, and German whereby said user may converse with said computer system in a plurality of languages allowing for the current invention to be accessible to a multitude of persons, allowing for increased access to medical information for non-English-speaking persons.

11. The method of claim 11 wherein said plurality of audio files to be played to said caller by said third means contains an audio message recorded by said caller's own doctor.

12. The method of claim 1 wherein said means for providing medical advice to said person further includes software as a means for transmitting medical advice over a data network.

13. The method of claim 12 wherein said software encodes transmitted data as HTML files.

14. The method of claim 12 wherein said software encodes transmitted data as VoiceXML files.

15. A method for enhancing a caller's experience of using an automated telephone service comprising:
a caller;
said caller's preferences including but not limited to voice of automated telephone service;
said caller's preferences stored in a computerized storage device;
a first means for identifying said caller;
a second means for loading said caller's preferences;
a third means for dynamically adapting said automated telephone service to said caller's preferences;
whereby such customization of automated telephone service increases said user's satisfaction of said automated telephone service.

16. A machine for providing medical advice over a telecommunications network comprising:
a display device providing the means for a human operator to view prompts and information necessary to practice the current invention;
an input device providing the means for the said operator to interact with the software component of the invention;
a processing unit for manipulation of data and for execution of software;
a memory which is used by said processing unit during said manipulation of data and execution of software;
a storage device for storage of the software and the files associated with the invention;
a first device for interfacing with said data network;
a second device for interfacing with a telecommunications system;
a first means for performing automatic speech recognition on an audio signal from second device;
a second means for synthesizing computer text into speech;
a human caller who is seeking medical advice, be they a patient or a caller acting as a proxy for said patient;
a third means which said caller can use to communicate with said computer system using spoken language using said second device and said first means;
said spoken language including but not limited to English, French, and German;
a plurality of audio files with prerecorded medical advice recorded by physicians;
a fourth means which allows for the playback of said audio files via said second device;
a fifth means for said caller to answer a series of questions, including but not limited to said patient's name, said caller's name, a phone number, a doctor's name, chief complaint, and a series of questions to determine if a high-risk medical condition exists, resulting in the selection of a subset of said audio files accessible by said computer system from a location selected from the group consisting of a local storage device, and a storage device located on a remote computer system accessible via said data network, to be played over said telecommunications network using said second device;
a sixth means which allows for said caller and said computer system interaction to be documented and saved in a storage device;
whereby the audio file played to said user via said telecommunications network contains medical information specific to said user's condition;
whereby said medical information is in said patient's doctor's own voice; and
whereby said patient will be more confident in said automated telephone service since a playback message was custom-tailored for said patient.

17. The machine of claim 16 wherein said data network is comprised of a network of type selected from the group consisting of the Internet, an intranet, and an extranet.
